(19) United States
(12) Patent Application Publication
(10) Pub. No.: US 2013/0245524 A1
(43) Pub. Date: Sep. 19, 2013

(54) KNEE ANKLE FOOT ORTHOSIS

(71) Applicant: THE GOVERNORS OF THE UNIVERSITY OF ALBERTA, Edmonton (CA)

(72) Inventor: Jonathon S. Schofield, Edmonton (CA)

(73) Assignee: THE GOVERNORS OF THE UNIVERSITY OF ALBERTA, Edmonton (CA)

(21) Appl. No.: 13/833,101

(22) Filed: Mar. 15, 2013

Related U.S. Application Data
(60) Provisional application No. 61/611,440, filed on Mar. 15, 2012.

(30) Foreign Application Priority Data
Mar. 15, 2012 (CA) 2771972

(51) Int. Cl. A61F 5/01

(52) U.S. Cl. CPC A61F 5/0125; A61F 5/0127
USPC 602/16; 602/26; 602/27

(57) ABSTRACT

A Knee-Ankle-Foot-Orthoses (KAFO) brace mechanically generates a knee extensor moment and allows for a flexed knee during STS, allowing for reduced upper body demand to be placed on the patient. An orthosis comprises a femoral brace and a tibial brace connected together with a pivot to form a knee joint between the femoral brace and the tibial brace, and a knee extension moment generator at the knee joint. Also, a foot brace may be connected to the tibial brace to form an ankle joint with an ankle plantar flexion moment generator disposed about the ankle joint.

### CROSS-REFERENCE TO RELATED APPLICATIONS

This application claims the benefit under 35 USC 119(e) of U.S. provisional application Ser. No. 61/611,440 filed Mar. 15, 2012.

### FIELD

Knee Ankle Foot Orthosis

### BACKGROUND

Knee-Ankle-Foot-Orthoses (KAFOs) are leg braces designed to assist in standing for patients with limited lower extremity function. The brace encompasses the thigh to the foot holding the knee extended and the ankle in a neutral position; thereby controlling balance and joint alignment (1). The intent of the brace is to provide stability and rigidity to the knee and ankle joints as a means of augmenting weight bearing capabilities (2). KAFOs have a variety of applications including: broken bones, arthritic joints, bowleg, knock-knee, knee hyperextension as well as muscular weakness and paralysis (1). Patients requiring KAFOs are often dependent on a wheelchair. Therefore, standing becomes an important physiological function with benefits including pressure relief, spasticity reduction, bowel and bladder management, among others (4). However, since a KAFO limits knee and ankle motion, rising from a chair becomes a significant challenge. Attempting to stand with straight knees, as compared to flexed knees, creates a larger standing force-moment lever arm between the ground and the patient's center of mass. As a result of the combination of this altered geometry and the inability to flex the knee (due to KAFO function and often physiologically), patients must adopt a modified Sit To Stand (“STS”) and Stand to Sit (“StandTS”) strategy. Typically STS while wearing a KAFO involves using fore arm crutches or a walker and substantial upper body strength to hoist oneself from seated position. Due to the user's inability to create a knee extensor moment, the patients will rely on their upper body strength to compensate and provide the anti-gravity moments to stand. Consequently, substantial demand is placed on the upper body and many KAFO users are unable to achieve STS independently. To understand the effect of removing the knee extensor moment during STS, non-pathological, or able-bodied, movements must first be understood. Current literature shows a wide variation in kinetic values associated with STS biomechanics. Peak knee extensor moment values have been reported in numerous studies with significantly large variations between them, ranging from 0.38 to approximately 1.0 Nm/kg (6)(7). In other words, the maximum values reported in current literature are approximately 260% the magnitude of the minimum reported values. Furthermore, no study has evaluated the biomechanics of the left and right leg independently over the entire STS cycle; the left and right side of each participant have been assumed to produce joint moment values symmetrically (6)(7) (8)(9). A possible explanation for this wide variation lies in the methods of estimating joint moment values. Many studies rely heavily on numerical modeling to try and reproduce movement patterns experienced during STS (8)(9). A second approach to quantify kinetic and kinematic is to use motion capture analysis and often inverse dynamics (10)(11). This method uses hemispherical markers in combination with motion capture cameras and force plates. Using inverse dynamic techniques and software, joint moment data can be calculated.

### SUMMARY

An orthosis comprising a femoral brace and a tibial brace connected together with a pivot to form a knee joint between the femoral brace and the tibial brace; and a knee extension moment generator disposed about the knee joint.

An orthosis comprising a tibial brace and a foot brace connected together with a pivot to form an ankle joint between the tibial brace and the foot brace; and an ankle plantar flexion moment generator disposed about the ankle joint.

An orthosis comprising a femoral brace and a tibial brace connected together with a pivot to form a knee joint between the femoral brace and the tibial brace, a knee extension moment generator disposed about the knee joint, a foot brace connected to the tibial brace to form an ankle joint and the knee extension moment generator being connected to provide an ankle plantar flexion moment generator deposed about the ankle joint.

Several options for providing the knee extension moment generator are proposed, the preferred design incorporating a pulley concentric to the knee joint and a cable extending over the pulley, the cable being operated by an actuator, for example a gas compression spring.

These and other aspects of the device and method are set out in the claims, which are incorporated here by reference.

### BRIEF DESCRIPTION OF THE FIGURES

Embodiments of a knee ankle foot orthosis will now be described with reference to the figures by way of example, in which:

FIGS. 1A-1D show four embodiments of a knee ankle foot orthosis.

FIG. 2 is a perspective view of an embodiment of a knee ankle foot orthosis with a cable and pulley system, and knee partially extended.

FIG. 3 is a perspective view of the knee ankle foot orthosis of FIG. 2 with the knee joint extended.

FIG. 4 is a perspective view of the knee ankle foot orthosis of FIG. 2 with knee joint flexed at 90 degrees.

FIG. 5 shows a KAFO with an ankle plantar flexion moment generator and the knee joint flexed at 90 degrees.

FIG. 6 shows a KAFO with an ankle plantar flexion moment generator and the knee joint extended.

### DETAILED DESCRIPTION

An orthosis is disclosed comprising a femoral brace and a tibial brace connected together with a pivot to form a knee joint between the femoral brace and the tibial brace; and a knee extension moment generator at the knee joint. A femoral brace is a brace that attaches to a person's upper leg and is connected for movement with the femur. A tibial brace is a brace that attaches to a person's lower leg and is connected for movement with the tibia. In another embodiment, and in like manner, an orthosis may comprise a foot brace connected to a tibial brace to form an ankle joint and an ankle plantar flexion moment generator at the ankle joint. A foot brace is a brace that attaches to a foot for movement with the foot. When a brace is connected for movement with a body member, the movement of the brace causes a corresponding movement of the body member. Any conventional design of brace may be used providing it is engineered to resist the forces developed by the knee extension moment generator. Suitable designs may be made of metal, fibre composites, plastic, combinations of these materials or other suitable materials. Parts of the knee extension moment generator may be used for the ankle plantar flexion moment generator.

The device is designed such that the force provided by the assistance mechanism is slightly lower than the weight of the individual. In addition, the assistance force supplied by the device is maximum at the “sit” position and minimum at the “stand” position. The force varies smoothly between those two positions which allows the individually to comfortably and safely achieve both sit-to-stand and stand-to-sit motions.

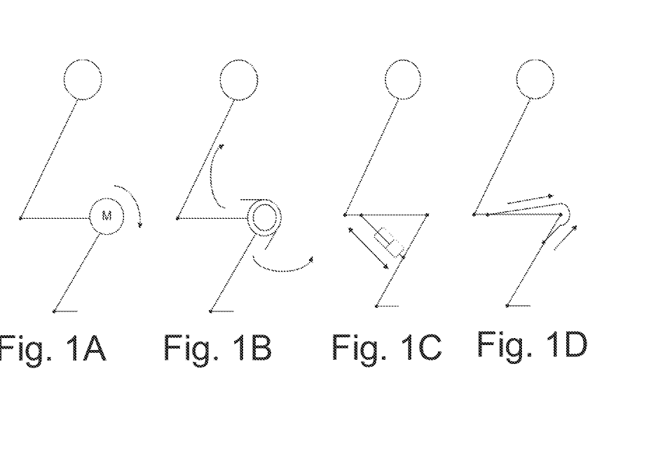

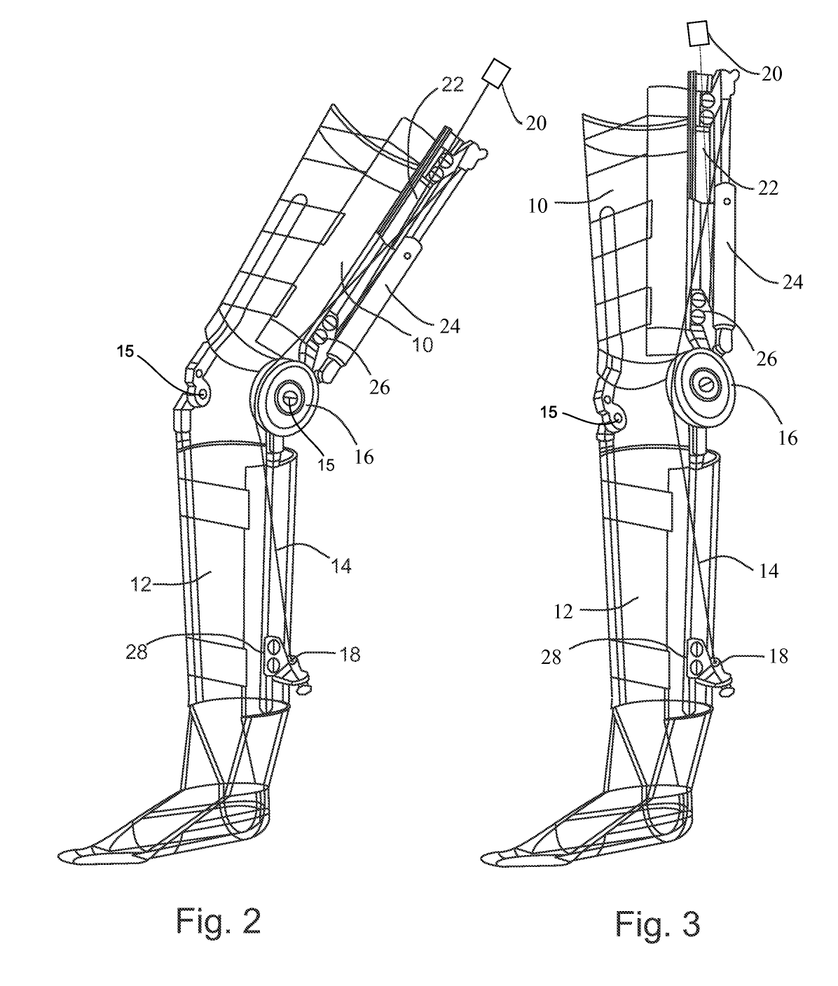

Referring to FIGS. 1A-1D there is shown four alternative embodiments of a knee ankle foot orthosis using various types of knee extension moment generators. In FIG. 1A, a motor and torque transmission device is placed concentric with the knee. In FIG. 1B, a torsional spring is placed concentric with the knee. In FIG. 1C, a linear actuator posterior is placed posterior to the knee. In FIG. 1D, a tensioned cable and pulley system to mimic quadricep force vectors is used. These moment generators may also be used as ankle plantar flexion generators when located at the ankle joint. In the case of the design in FIG. 1D, the same force generator, a gas compression spring, may be used for the both the knee extension moment generator and ankle plantar flexion generator. The knee extension moment generators of FIGS. 1A-1D are disposed about the knee joint as follows. In FIG. 1A, a motor is provided that is anchored to both braces and rotates about an axis preferably aligned with the knee joint. In FIG. 1B, the torsional spring is attached to both braces and also is preferably concentric with the knee joint. In FIG. 1C, the linear actuator is anchored to both braces on either side of the knee joint. The embodiment of FIG. 1D is discussed in more detail in relation to FIGS. 2-4.

An exemplary KAFO shown in FIG. 2 comprises a femoral brace 10, tibial brace 12, cable 14, pulley 16, cable anchor 18, push button 20, guide block 22, gas compression spring 24, and anchor brackets 26. The anchor brackets 26 anchor the gas compression spring 24 to the femoral brace 10. Various methods may be used to anchor the gas compression spring 24 to the anchor brackets 26, or the gas compression spring 24 may be made integral with the femoral brace 10. In some embodiments, the gas compression spring 24 may be anchored to the tibial brace 12, or there may be gas compression springs or other force generators on both the tibial brace 12 and the femoral brace 10.

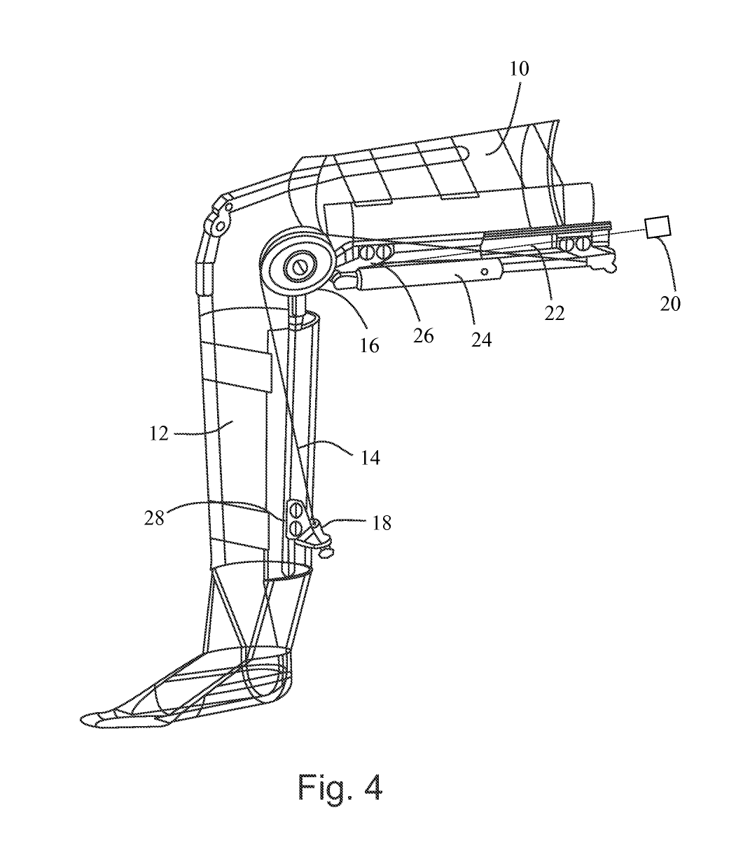

The KAFO is illustrated in FIG. 3 with the knee joint extended, and in FIG. 4 with the knee joint flexed at 90 degrees. The femoral brace 10 and tibial brace 12 are connected together with a pivot 15 to form a knee joint between the femoral brace 10 and the tibial brace 12. A knee extension moment generator is provided at the knee joint from the pulley 16, combined with the cable 14 and gas compression spring 24. With extension of the gas compression spring 24, which may be remotely triggered by any suitable means, tension will be created in the cable 14. This tension will cause an extension moment in the knee hinge 15 of the KAFO and assist sit-to-stand movements. Inversely the system will resist knee flexion during stand-to-sit movements. The resistance will allow for a controlled descent back into the chair and ‘reload’ the system.

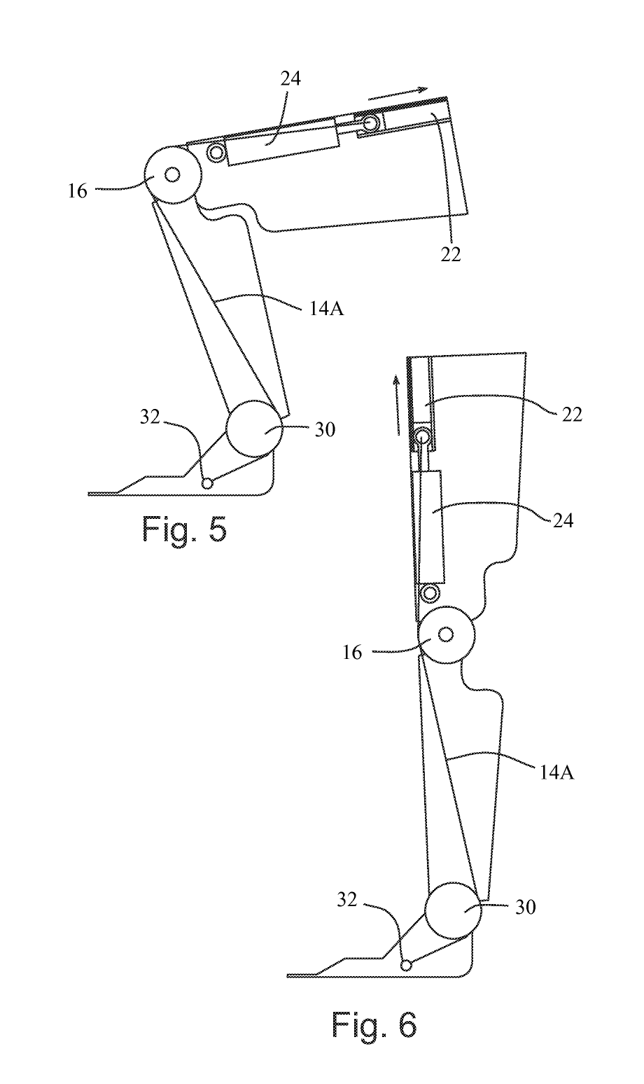

As shown in FIGS. 5 and 6, an example of an embodiment with power assisted ankle plantar flexion is disclosed. The embodiment shown in FIGS. 2-4 may for example be modified to allow for ankle plantar flexion, or ankle plantar flexion may be provided on a separate device. In a separate ankle plantar flexion orthosis, the gas compression spring or other force generator, may be attached to the tibial brace and connected directly to a cable that extends around a pulley at the ankle joint to an anchor point on the foot brace. The combined orthosis shown in FIGS. 5 and 6 may use an appropriately sized gas compression spring 24 and single cable 14A. An additional cable guide or pulley system is provided at the ankle joint, with the ankle pulley 30 preferably concentric to the ankle joint. The KAFO orthotic in this embodiment incorporates a hinged ankle joint (ankle hinge systems are commercially available). The ankle pulley 30 (cable guide) is positioned such that the cable 14A passes posteriorly to the ankle joint's center of rotation. The cable 14A is anchored distally from the ankle joint at a second cable anchor 32.

When the gas compression spring is actuated tension are created in the cable 14A. Due to the pulley 16 (or other cable guide) at the knee and ankle pulley 30 (or other cable guide) at the ankle joints, joint moments are created at these locations. The direction the cable is warped will create knee extension and ankle plantar flexion using a single gas compression spring and single cable. Similar to the knee-extension-only design, actuation of the device can be accomplished remotely, and the system will be able to support the user statically mid sit-to-stand movement due to the self-locking nature of the gas spring. The ankle moment generator may use any of the modifications shown in FIGS. 1A-1D and discussed below.

Alternatives for any of the disclosed embodiments of the Gas Compression Spring include a mechanical spring, electric linear actuator and controller, hydraulic cylinder with reservoir, pump and valves, or pneumatic cylinder with reservoir, pump and valve. Alternatives for the pulley and ankle pulley include a bracket with radius and guide groove, cable, belt, rope and chain. Alternatives for activation of the force generator include a push button, lever, switch or solenoid.

Quantification of the biomechanical forces in healthy STS movements using motion capture analysis has shown that an orthosis of the types disclosed here will work as an assistive sit-to-stand knee ankle foot orthosis.

The kinematics and kinetics of ten participants' STS movements were quantified at the Glenrose Rehabilitation Hospital's Motion Lab (GRH). Ethics approval was obtained through the University of Alberta ethics review board. Participants were recruited within the University of Alberta Civil Engineering Department. The participants selected were males between the ages of 21 and 35 years (mean: 25, SD: 4). Males were specifically selected to remove the variation in weight distribution typically seen between females and males; furthermore, all subjects reported having no prior injuries, pathologies, or conditions that may affect their STS movements.

Eighteen reflective hemispheres (1.5 cm diameter) were used to define eight body segments representing the participant's feet, shanks, thighs, pelvis and torso (12). Lower extremity markers were positioned according to a modified Helen Hayes marker set protocol (13). Markers were positioned on both the left and right side at the: anterior superior iliac spine, lateral and medial epicondyle of each knee, lateral and medial malleolus, second metatarsal head, and calcaneus. Wand style markers were positioned for redundancy along the tibial and femoral axis. A single marker was positioned at the sacrum, and three upper body markers were positioned at C7, centered between the clavicles, and centered on the sternum.

Marker position was captured using eight motion cameras and an Eagle Digital Motion Analysis system sampling at 120 Hz. Two AMTI force plates sampling at 2400 Hz were utilized to capture ground reaction forces. Subjects were instructed to fold their arms across their chest and rise 10 times, at a self-selected pace, from a backless, armless, 48 cm tall chair (14). The chair was positioned such that the participant could comfortably place one foot on each force place. The trial was assumed to begin at the onset of hip flexion, i.e. the mass transfer phase, and end when extension motion ceased in the hip, knee and ankle (15).

EVaRT 5 software was utilized to virtually join markers and pre-process the raw motion data. This data was imported into C-Motion's Visual 3D software to perform an inverse dynamic analysis. Within Visual 3D, a general three dimensional model body was scaled to each participant's motion data. Body-segment rotational properties were input according to 50th percentile anthropometric data (16) (17). Algorithms within the software performed inverse dynamic calculation based on these input data. To remove electronic noise from marker position data, a fourth-order, zero phase-shift Butterworth filter was utilized in Visual 3D. The filter was set to attenuate noise over a frequency of 4 Hz while allowing data under this threshold (typical of human motion) to pass unaffected (17).

Peak knee joint moments were determined for each leg, of each participant, of each STS trial using the output data from Visual 3D. In total, 200 peak knee moments were quantified and normalized by each participant's body mass. These, normalized peak knee moments were averaged to represent the mean knee joint moment developed during STS from the ten able-bodied participants. This resulting mean knee joint extensor moment value was used as the target value to be provided to patients and consequently to guide the development of the assistive STS KAFO prototype.

Through collaboration with physical therapists and orthotists at the GRH, the four conceptual designs shown in FIGS. 1A-1D for an assistive KAFO were proposed. Each conceptual design uses a different method to compensate lower extremity weakness by mechanically generating a knee extension moment in the KAFO knee joint. The STS trials were used to develop a target value for the maximum (peak) knee joint moment each design must develop. With this additional knee moment, the need for maintaining extended knees in the locked KAFO position during STS is eliminated; thereby, reducing the upper extremity moment required. Kinetically, flexed knees during STS reduces the moment arm a KAFO user, with straight legs, must overcome. Furthermore, introducing a knee extension motion assists achieving knee extension, a crucial component of rising from a chair that is absent in most KAFO STS strategies. As a result, the moment that must be created at the shoulder of the patient should be dramatically reduced.

Eleven criteria, pertinent to the design of the prototype, were identified and weighted according to importance by engineers, and clinicians at the University of Alberta and Glenrose Rehabilitation Hospital. These criteria included affordability, reliability, and weight among others. They were then weighted based on their importance to a successful mechanical design as well as to end user acceptance. The values ranged from 1, indicating very little importance, to 3, indicating very high importance, respectively. Each conceptual design was then rated on its ability to meet these eleven criteria. Again, a weighting system was used. This system used conformance values between 0 indicating an inability to meet the criteria and 1 a very strong ability to meet the criteria, respectively. A Pugh Matrix was used to sum the weighted criteria and ultimately determine the most appropriate design (18). A total summed score of 29 would be an ideal candidate and a score of zero would have no ability to meet the design criteria.

TABLE 1
A Pugh Matrix to weight relevant design criteria and each conceptual
design's ability to meet these criteria

| | Importance | Linear Actuators | | Torsion Springs | | Electric Motors | | Tension Cables | |
|---|---|---|---|---|---|---|---|---|---|
| | (3-Very, 1-Low) | Conformance | Score | Conformance | Score | Conformance | Score | Conformance | Score |
| Quiet actuation | 1 | 0.5 | 0.5 | 1 | 1 | 0.5 | 0.5 | 1 | 1 |
| Small - medial | 3 | 0.75 | 2.25 | 0.25 | 0.75 | 0.25 | 0.75 | 1 | 3 |
| lateral profile | | | | | | | | | |
| Light weight | 3 | 0.75 | 2.25 | 0.5 | 1.5 | 0.25 | 0.75 | 1 | 3 |
| Affordable | 2 | 0.75 | 1.5 | 1 | 2 | 0.5 | 1 | 1 | 2 |
| Reliable- simplicity | 3 | 0.75 | 2.25 | 1 | 3 | 0.75 | 2.25 | 0.5 | 1.5 |
| Durability | 3 | 0.75 | 2.25 | 0.5 | 1.5 | 0.75 | 2.25 | 0.75 | 2.25 |
| Easy maintenance | 2 | 0.5 | 1 | 1 | 2 | 0.5 | 1 | 0.75 | 1.5 |
| Manufacturability | 2 | 0.75 | 1.5 | 0.75 | 1.5 | 0.75 | 1.5 | 0.75 | 1.5 |
| Mechanical control - | 3 | 0.5 | 1.5 | 0 | 0 | 1 | 3 | 0.5 | 1.5 |
| velocity, forces, etc | | | | | | | | | |
| No external power | 2 | 1 | 2 | 1 | 2 | 0 | 0 | 1 | 2 |
| Source Required? | | | | | | | | | |
| Low impact of | 2 | 1 | 2 | 0.75 | 1.5 | 0.5 | 1 | 0.75 | 1.5 |
| system failure | | | | | | | | | |
| Aesthetically pleasing | 3 | 0.5 | 1.5 | 0.5 | 1.5 | 0.25 | 0.75 | 0.5 | 1.5 |
| Total Compliance | | | 20.50 | | 18.25 | | 14.75 | | 22.25 |
| Score | | | | | | | | | |

Once an ideal candidate was selected, a three dimensional model of the prototype was created using Dassault Systemes' SolidWorks. This model allowed for a visual representation of the model as well as creation of part and assembly drawings. The parts utilized in the final design were manufactured using a donated KAFO brace, a local water jet cutting vendor as well as off-the-shelf parts.

The results of the Pugh matrix indicated that the tensioned cable design was the most appropriate to meet the design criteria outlined (Table 1). This design uses a remote triggered locking-gas-compression spring positioned longitudinally along the femoral portion of the KAFO brace. When the spring extends, it drives a guide-block and create tension in the attached cable. Since the cable is anchored to the tibial frame and passed over a pulley positioned concentric to the KAFO knee joint, this tension generates an extensor moment at the knee.

The results of the STS motion analysis provided two useful pieces of information for the prototype design. First, healthy subjects typically produce noticeable asymmetrical peak moment development at the knee joint over the STS cycle. This finding is contrary to the typical assumption of symmetry made in most current STS studies (8)(9). Peak values in the left and right leg could be averaged for each participant, and percent difference calculations conducted on these average values for each participant's left and right side. The participant with the maximum deviation from their average was produced a 13.41% deviation and the minimum participant's value was calculated at 2.84% (Mean: 7.22%, SD: 0.08).

Second, the values of the peak knee extensor moment provided the necessary peak torque required by the prototype. Ten STS cycles of ten participants' two legs were evaluated, producing data for 200 peak knee joint moments. For the development of the assistive prototype, the average peak moment of these 200 data sets served as the target value to design to. The inverse dynamic analysis performed on the motion capture data yielded average peak knee moments for each participant between 0.50 and 0.93 Nm/kg-body mass (mean: 0.71 Nm/kg-BM, SD: 0.14). Therefore, the mean value of 0.71 Nm/kg was used to guide the design of the KAFO prototype for a 90 kg individual. As a result the assistive mechanism must create approximately 63 Nm of torque at the knee joint.

TABLE 2
Peak knee moments for each of the ten participants
and the overall average values

| | Average | | Normalized |
|---|---|---|---|
| | Peak Knee | Body | Peak Knee |
| | Moment | Mass | Moment |
| Participant | (Nm) | (kg) | (Nm/kg) |
| 1 | 47.16 | 70 | 0.67 |
| 2 | 58.98 | 76 | 0.78 |
| 3 | 40.55 | 73 | 0.56 |
| 4 | 46.11 | 74 | 0.62 |
| 5 | 54.96 | 68 | 0.81 |
| 6 | 72.21 | 79 | 0.91 |
| 7 | 66.13 | 71 | 0.93 |
| 8 | 34.81 | 49 | 0.71 |
| 9 | 49.59 | 79 | 0.63 |
| 10 | 33.11 | 66 | 0.50 |
| Overall | 50.36 | 70.50 | 0.71 |
| Average | | | |
| SD | 12.85 | 8.71 | 0.14 |

The first-prototype was machined to utilize a 900-450 Newton gas compression spring to drive a cable tensioning system. The assistive system can be easily removed and added to most pre-existing KAFO designs with minor modifications. The prototype can be remote triggered by the user to drive knee extension.

Calculations have been performed on the current design to determine the moment (torque) output. Using the as-built geometry of the prototype, the effective moment arm can be calculated at various positions of knee extension. When coupled with the force curves of the gas compression spring, the theoretical torque development of the KAFO was plotted. Referring to a peak torque of approximately 63 Nm and the 0.71 Nm/kg average peak moment value from the STS trials indicates that the as-built device can provide peak torque equivalent to that required by a 90 kg patient during STS. Furthermore, the simplicity of the design allows for flexibility in performance characteristics of the device. Torque of the prototype can be adjusted in three ways. A tension adjustment system was incorporated in the design to accommodate fine tuning of the KAFO knee moment. Moderate torsional adjustment can be accomplished through altering the geometry of the pulley mounting bracket. And finally changing the model of gas compression spring can allow for dramatic changes in torque development of the prototype.

A prototype has been developed that can provide sufficient torque to assist in STS of KAFO dependent patients. Clinic-based testing must still be conducted for commercial use. For example, it is desirable that the timing of torque development in the orthosis match a healthy STS cycle. Matching may be achieved through study of an STS cycle for example using motion capture analysis. The force generator may be designed to match the healthy STS forces. Once a KAFO dependent participant can achieve STS, minimizing size and weight of the assistive device will also be desirable.

Using motion capture analysis, the peak knee joint extensor moments were quantified in 10 participants. These values were utilized as target design values in the development and manufacturing of the first assistive STS prototype. This device appears to have the potential to be successful in assisting STS in subjects prescribed KAFOs. The ability for the assistive prototype to meet the torque demands of a KAFO user will be addressed in future testing. Based on the results to date it can be soundly predicted that the KAFO devices proposed here may be utilized by a wide spectrum of users.

Gas spring: a type of spring that, unlike a typical metal spring, uses a compressed gas, contained in a cylinder and compressed by a piston, to exert a force.

An embodiment of an orthosis preferably utilizes a gas compression spring to generate a knee extension moment. Gas compression spring technology may not be as widely known as mechanical springs; however, they allow for exceptional versatility and flexibility in the KAFO. Arguably the use of a mechanical spring may achieve the same function; however, the KAFO would lose certain adjustment and functional aspects.

A mechanical spring would have to be compressed when the KAFO client is seated and the device is not in use. The compressed springs would store a substantial amount of potential energy in close proximity to the client's body. If the compression mechanisms of the device were to fail, this stored energy has the potential to rapidly release. This rapid decompression of the springs will have the potential to create projectiles, pinch-points or other safety concerns. The weakest mechanical point in a gas compression spring is the seals inside the gas cylinder. If a gas spring were to fail, one would expect compressed gas to flow past the seals internally in the spring. Ultimately failure in a gas compression spring would cause the pressure inside the spring to reach equilibrium. This would result in the spring being unable to extend or retract. This form of failure poses minimal to no risk to the KAFO client.

The magnitude of the knee extension moments required during STS and StandTS, vary based on weight, height and other physiological factors of the client. Therefore it is desirable that the KAFO be able to accommodate a spectrum of users and consequently output knee extension moments. A mechanical spring will generate force based on displacement. Typically these springs will not allow for adjustment of force values. In terms of the KAFO, to change the output knee moment, a different set of mechanical springs would have to be used for each client. Gas compression springs; however, generate force based on gas pressure. Many commercially available designs come with bleed-off valves. These valves will allow for pressure in the spring to be released such that a desired output force value is achieved. For the KAFO, this would allow the orthotist to ‘tune’ each gas spring to the appropriate value for each client, rather than replacing the spring itself.

Mechanical springs use material deformation to generate force. Typically larger sized springs with more material will generate more force. As a result the weight and size of a mechanical spring will be related to its force output. Consequently, to generate the force values required by the KAFO, either a bulky single spring must be used or multiple smaller springs. Gas compression springs utilize gas pressure to create linear force. Gas springs with higher force outputs, tend to use higher gas pressures. This results in higher output force with minimal mass increase. Relative to mechanical springs, for force values typical of those required by the KAFO, a gas compression spring will yield a more desirable weight to force and size to force ratio.

In the application of the KAFO, gas compression springs are a much more versatile tool than mechanical spring. Several types of gas compression springs exist. The KAFO utilizes a locking spring. This spring allows for spring extension (and ultimately knee extension) to be stopped and held at any position along its stroke. Using a mechanical spring to do this would not be possible without designing an accessory mechanism separate from the spring. Furthermore, extension of the gas spring can be triggered through a variety of ways (Push button, levers, solenoids, etc.). Again a mechanical spring requires a separate mechanism to ‘lock’ the spring in place when extension is not desired. Like mechanical springs, gas compression springs can be custom ordered, to best fit the client, from a multitude of suppliers. As a result, a gas compression spring allows for a much more versatile actuation device that can be tailored to a client's individual need with only minor adjustments.

Function of the Gas Spring: The design utilizes a locking gas compression spring to drive a linear guide block; both components are mounted to the femoral brace of the KAFO. A cable is anchored to the guide block, passed over a pulley positioned non-concentrically with the knee hinge, and anchored to the tibial portion of the KAFO. By driving the guide block, tension will be created in a cable which will create a knee extension moment during STS and create resistance to knee flexion during stand-to-sit. The system will be push-button or remote trigger-operated by the user.

Function Explanation: With extension of the remotely triggered gas compression spring, tension will be created in the cable (indicated by the solid red line). This tension will cause an extension moment in the knee hinge of the KAFO and assist sit-to-stand movements. Inversely the system will resist knee flexion during stand-to-sit movements. The resistance will allow for a controlled descent back into the chair and ‘reload’ the system.

### REFERENCES

1. KAFO (Knee Ankle Foot Orthosis). Sheck and Siress. s.1.: Sheck & Siress Prosthetics Orthotics Pedorthotic, 2011.
2. Ambulatory KAFOs: A Physiatry Perspective. Herbert, J. 3S, s.1.: JPO, 2006, Vol. 18, p. 169.
3. O'Connor Sean. Transabled.org [Online] April 2007. http://transabled.org/thoughts/braces-and-my-relationship-to-them.htm
4. AAOS atlas of orthoses and assistive devices. Hsu, J., Michael, J. and Fisk, J. 4, s.1.: American Academy of Orthopaedic Surgeons, 2008.
5. Comparison of functional electrical stimulation to long leg braces for upright mobility for children with complete thoracic level spinal injuries. Bonaroti, D., et al., et al. 9, s.1.: Archives of Physical Medicine and Rehabilitation, 1999, Vol. 80, pp. 1047-1053.
6. Biomechanical analysis of relationship between movement time and joint moment development during sit-to-stand task. Yoshioka, S., et al., et al. 27, s.1.: Biomedical Engineering Online, 2009, Vol. 8, pp. 1-9.
7. Biomechanical Analysis of Sit-to-Stand Movement in Normal and Obese. Sibella, F., et al., et al. 5, s.1.: Clinical Biomechanics, 2003, Vol. 18, pp. 745-750.
8. Dynamics of sit-to-stand movement. Roberts, P D. and McCollum, G. s.1.: Biological Cybernetics, 1996, Vol. 74, pp. 147-157.
9. Kinemantics of Sagittal Spine and Lower Limb Movements in Healthy Older Adults During Sit-to-Stand from Two Seat Heights. Kuo, Y., Tully, E. and Galea, M. 1, s.1.: SPINE, 2009, Vol. 35.
10. On the Assumption of Bilateral Lower Extremity Join Moment. Lundin, T M., Grabiner, M D. and Jahnigen, D W. 1, s.1.: Journal of Biomechanics, 1995, Vol. 28, pp. 109-112.
11. Symmetry of ground reaction forces and muscle activity in asymptomatic subjects during walking, sit-to-stand, and stand-to-sit tasks. Burnett, D., et al., et al. s.1.: Journal of Electromyography and Kinesiology, 2011, pp. 610-615.
12. Can hip and knee kinematics be improved by eliminating thigh markers? Schulza, B. and Kimmelb, W. 7, s.1.: Clinical Biomechanics, 2010, Vol. 25, pp. 687-692.
13. Group, Biomechanics Research. Marker Placement Protocol. Life Modeler. [Online] http://www.lifemodeler.com.
14. Sit-to-Stand Movement Pattern A Kinematic Study. Nuzik, S., et al., et al. 11, s.1.: Physical Therapy, 1986, Vol. 66, pp. 1708-1713.
15. Effects of Ageing on Quadriceps Muscle Strength and on the Forward Shift of the Center of Pressure During Sit-to-Stand Movement from a Chair. Miyoshi, K. and al., et. s.1.: Journal of Physical Therapy Sciences, 2005, Vol. 17, pp. 23-28.
16. Zatsiorsky, V. Kinetics of Human Motion. 1. s.1.: Human Kinetics, 2002.
17. Winter, David A. Biomechanics and Motor Control of Human Movement. [ed.] Second. s.1.: John Wiley & Sons, Inc., 1990. 0-471-50908-6.
18. Process Excellence. The Pugh Matrix. Process Management and Analysis. [Online] 2011.

Immaterial modifications may be made to the embodiments described here without departing from what is covered by the claims. In the claims, the word “comprising” is used in its inclusive sense and does not exclude other elements being present. The indefinite articles “a” and “an” before a claim feature do not exclude more than one of the feature being present. Each one of the individual features described here may be used in one or more embodiments and is not, by virtue only of being described here, to be construed as essential to all embodiments as defined by the claims.

What is claimed is:

1. An orthosis, comprising:
a femoral brace and a tibial brace connected together with a pivot to form a knee joint between the femoral brace and the tibial brace; and
a knee extension moment generator disposed about the knee joint.

2. The orthosis of claim 1 in which the knee extension moment generator comprises a pulley concentric to the knee joint and a cable extending over the pulley, the cable being operated by an actuator.

3. The orthosis of claim 2 in which the actuator comprises a gas compression spring.

4. The orthosis of claim 2 in which the actuator is anchored to the femoral brace.

5. The orthosis of claim 3 in which the actuator is anchored to the femoral brace.

6. The orthosis of claim 1 in which the knee extension moment generator comprises a motor and torque transmission device concentric with the knee joint.

7. The orthosis of claim 1 in which the knee extension moment generator comprises a linear actuator posterior to the knee joint.

8. The orthosis of claim 1 in which the knee extension moment generator comprises a torsional spring concentric with the knee joint.

9. The orthosis of claim 1 in which the knee extension moment generator comprises a tensioned cable and pulley system to mimic quadricep force vectors.

10. The orthosis of claim 1 further comprising a foot brace connected to the tibial brace to form an ankle joint and the knee extension moment generator being connected to provide an ankle plantar flexion moment generator disposed about the ankle joint.

11. The orthosis of claim 1 in which the knee extension moment generator comprises a pulley concentric to the knee joint and a cable extending over the pulley, the cable being operated by an actuator.

12. The orthosis of claim 11 further comprising an ankle pulley concentric to the ankle joint, the cable extending over the ankle pulley, and the cable being anchored to the foot brace.

13. The orthosis of claim 12 in which the actuator comprises a gas compression spring.

14. The orthosis of claim 13 in which the actuator is anchored to the femoral brace.

15. The orthosis of claim 12 in which the actuator is anchored to the femoral brace.

16. The orthosis of claim 10 in which the knee extension moment generator comprises a motor and torque transmission device concentric with the knee joint, and the ankle plantar flexion moment generator comprises an ankle torque transmission device.

17. The orthosis of claim 1 in which the knee extension moment generator comprises a linear actuator posterior to the knee joint.

18. An orthosis comprising a tibial brace and a foot brace connected together with a pivot to form an ankle joint between the tibial brace and the foot brace; and an ankle plantar flexion moment generator disposed about the ankle joint.

19. An orthosis comprising a femoral brace and a tibial brace connected together with a pivot to form a knee joint between the femoral brace and the tibial brace, a knee extension moment generator at the knee joint, a foot brace connected to the tibial brace to form an ankle joint and the knee extension moment generator being connected to provide an ankle plantar flexion moment generator disposed about the ankle joint.
